(19) United States
(12) Patent Application Publication
(10) Pub. No.: US 2026/0258135 A1
(43) Pub. Date: Sep. 3, 2026

(54) ANTIBODY AFFINITY MATURATION METHOD, ANTI-HUMAN PD-L1 SINGLE DOMAIN ANTIBODY AND USE THEREOF, NUCLEIC ACID, RECOMBINANT VECTOR, TRANSFORMANT, AND PHARMACEUTICAL COMPOSITION

(71) Applicant: Biointron Biological Inc., Shanghai (CN)

(72) Inventors: Yufang WANG, Shanghai (CN); Haisong LU, Shanghai (CN); Chuan LIU, Shanghai (CN); Jingqiu TANG, Shanghai (CN)

(73) Assignee: Biointron Biological Inc., Shanghai (CN)

(21) Appl. No.: 18/995,831

(22) PCT Filed: Feb. 14, 2023
(86) PCT No.: PCT/CN2023/075819

§ 371 Date: Jan. 17, 2025

(30) Foreign Application Priority Data
Sep. 27, 2022 (CN) 202211182066.8

(51) Int. Cl. C07K 16/28; C07K 1/22; G01N 33/68

(52) U.S. Cl. CPC C07K 16/2827; C07K 1/22; G01N 33/6854; C07K 2317/569

(57) ABSTRACT

Provided are an affinity maturation method, an anti-human PD-L1 single domain antibody and a use thereof, a nucleic acid, a recombinant vector, a transformant, and a pharmaceutical composition. The antibody affinity maturation method of the present invention is a method combining full-coverage single-point saturation mutagenesis on CDRs and an ultrahigh-throughput expression screening system of a mammalian system, and unbiased full-coverage mutagenesis is carried out on amino acids in the CDRs of an antibody by means of an equal-ratio primer mixture.

### TECHNICAL FIELD

The present invention belongs to the field of biomedicine and antibody engineering, and in particular, relates to an antibody affinity maturation method.

### DESCRIPTION OF RELATED ART

Currently, the development of antibodies mainly originates from mouse hybridoma technology and in vitro antibody library technology. Candidate antibodies are initially obtained through antigen design and antibody screening, and then antibodies with application value are finally obtained through a series of related biological property tests and functional verifications. In the actual development process, there is a need to improve many properties of antibodies obtained via conventional screening methods, such as affinity, immunogenicity, half-life, etc., among which antibody affinity maturation is one of the most important improvement directions. The development of antibody affinity maturation technology will not only help improve the specificity and potency of antibodies, reduce the dosage of antibody drugs, and reduce toxic side effects, but also help people better understand the mechanism of interaction between antibodies and targets and better understand the function of targets.

In recent years, with the gradual deepening of human research on in vivo antibody affinity maturation and the rapid rise of antibody engineering research, in vitro antibody affinity maturation research has gradually become a research hotspot. In vitro antibody affinity maturation belongs to the category of evolution of functional protein molecules in vitro. Its research strategies are mostly proposed based on the understanding of the laws of antibody affinity maturation in vivo, and most of them simulate the way antibody affinity maturation occurs in vivo. Currently, the following are mainly used:

The error-prone PCR method is used to introduce point mutations. The key to this method is how to choose a suitable mutation frequency. Generally, the frequency of beneficial mutations is very low, and the vast majority of mutations are harmful. If the mutation frequency is too high, it will be almost impossible to screen out beneficial mutations; and the mutation frequency should not be too low, otherwise the wild type without any mutation will become the dominant type in the mutant population, and it will also be difficult to screen out ideal mutants.

DNA shuffling technology is to use deoxyribonuclease I to cut homologous antibody genes into fragments no longer than 50 bp, which are then randomly combined and subjected to PCR amplification. It includes the processes of random cutting, recombination and screening of antibody fragments, which simulates the affinity maturation process of natural antibodies to a certain extent and accelerates the speed of in vitro directed evolution.

CDR region recombination takes advantage of the structural characteristics that the constant region of antibodies is highly conserved, and in the variable region, only the CDR region closely related to antigen binding shows high variability, while other parts are also relatively conservative, and proposes the strategy of only focusing on mutations (mainly random mutations) in the CDR region of antibodies to modify the affinity of antibodies, which has achieved good effect.

Chain shuffling is also called chain replacement technology. This is a very simple in vitro affinity maturation technology for antibodies. Based on the principle of random pairing of variable regions of antibodies, one chain of the antibody is kept unchanged and the other chain is replaced to screen for high-affinity antibody molecules. However, the disadvantages of this technology are also obvious: it requires a relatively clear understanding of antigens and this technology can only be used on the basis of a relatively complete primary antibody library or antibody.

PD-1 stands for programmed death receptor 1, which is an important immunosuppressive molecule and a member of the CD28 superfamily. PD-L1 stands for programmed death receptor-ligand 1, which is a type I transmembrane protein with a size of 40 kDa. PD-1 is located in T cells and can bind to PD-L1 in stromal cells. The binding of the two acts as a co-inhibitory signal that mediates T cell activation to inhibit the killing function of T cells and negatively regulate the human immune response. Therefore, disrupting the interaction between PD-L1/PD-1 shows great potential in releasing the immune system's killing power against cancer cells.

PD-1-PD-L1 immunotherapy is a new generation of anti-tumor therapy that is currently attracting much attention. It aims to use the body's own immune system to fight tumor cells. The recognition of PD-1 and PD-L1 is blocked through PD-1 or PD-L1 antibodies, thereby restoring the normal function of T cells to recognize and defend against attacks and killing tumor cells. To date, 7 immune checkpoint inhibitors for the PD-1/PD-L1 pathway have been approved by the FDA: 4 anti-PD-1 monoclonal antibodies and 3 PD-L1 monoclonal antibodies, including the PD-1 monoclonal antibody Dostarlimab approved in April 2021. A report titled “Challenges and opportunities in the PD1/PDL1 inhibitor clinical trial landscape” published in Nature review on Feb. 10, 2022 updated the latest status of PD-1/PD-L1 clinical trials, including the use of clinically approved treatments and a summary of emerging patterns.

However, during clinical use, it has been found that although PD-1/PD-L1 therapy is effective, individual differences are very large. Some patients have achieved significant results, but some patients have no effect. And, similar to other cancer therapies, it also has side effects, which can be serious and even life-threatening. This is usually caused by an overactive autoimmune response. Increasing the affinity of PD-L1 single domain antibodies will increase the specificity of PD-L1 single domain antibodies, and high-affinity PD-L1 single domain antibodies will improve the effects in tumor treatment and detection.

### SUMMARY

The objectives of the present invention are to overcome the shortcomings of the prior art, to improve and upgrade the existing mutation technology so as to take advantage of its strengths and avoid its weaknesses, and to establish a highly efficient and simpler antibody affinity maturation system by using unbiased, full-coverage single-point directed mutation technology in the CDRs and combining it with a mammalian high-throughput antibody expression system.

Technical solution of the present invention is as follows:

In a first aspect of the present invention, an antibody affinity maturation method is provided, wherein the method can screen out antibodies with high affinity.

The method involves single-point saturation mutation technology and mammalian high-throughput expression, wherein the single-point saturation mutation technology uses a mixture of equal proportions of primers to perform unbiased, full-coverage single-point saturation mutation on all amino acids in the CDR region of the antibody.

In the present invention, combining the mutation technology with no bias, that is, 18 kinds of amino acids (20 kinds of amino acids excluding themselves and cysteine, the disulfide bond produced from cysteine can affect the subsequent processes) appear with equal probability, and full coverage, that is, all amino acids in the CDR region of the antibody are mutated, with the mammalian high-throughput antibody expression technology is expected to establish a more efficient and simpler antibody affinity maturation system.

In particular,
the affinity maturation method of the present invention, called FCMES-AM, is specifically a method
that combines single-point saturation mutation with full coverage of the CDR region with a mammalian ultra-high throughput expression screening system.

Furthermore, the method for single-point saturation mutation with full coverage of the CDR region is specifically a method
for performing unbiased, full-coverage mutation on each amino acid in the CDR region of the antibody by using a mixture of equal proportions of primers.

Furthermore, the method of the mammalian ultra-high throughput expression screening system comprises the following specific steps: 1) simultaneously expressing thousands of microwells using 96-well cell culture plates with each well expressing only a single antibody with one amino acid mutation to cover all mutation points in the CDR region; and 2) performing affinity screening on all expression supernatants using an affinity screening ELISA method to obtain high-affinity mutation hotspots.

Furthermore, expression screening cells used in the method of the mammalian ultra-high throughput expression screening system are mammalian expression cells, which eliminates the influence of display system proteins on antibody detection in conventional display systems.

Mammalian expression has many advantages in terms of protein folding, post-translational modification, and codon preference, which also makes the antibodies expressed in the mammalian system have the same or similar modifications as human antibodies. In addition, the antibodies expressed in the mammalian system are secreted into the supernatant and exist independently from the cells, which also eliminates the influence of display system proteins on antibody detection in conventional display systems.

Furthermore, the affinity screening ELISA method is specifically an ELISA method by capturing antibodies in the expression supernatants uniformly to eliminate the influence of concentration differences, thereby reflecting affinity differences through color development differences.

Furthermore, the method for antibody affinity maturation of the present invention includes the following steps:
1) constructing and verifying mutants of the antibody by the single-point saturation mutation technique;
2) recombining the mutants into an expression vector;
3) performing high-throughput extraction of plasmids and transfection of mammalian cells for expression;
4) screening mutation hotspots by orthogonal ELISA and sandwich ELISA;
5) performing hotspot combination by error-prone PCR;
6) performing relative affinity ranking of antibodies of hotspot combination by sandwich ELISA; and
7) selecting antibodies with improved affinity for expression and affinity detection.

Furthermore, the step 1) comprises constructing mutants of all amino acids in the CDRs of the antibody, and the specific process of single-point saturation mutation of each amino acid is as follows: amplifying an antibody gene fragment A before a mutation point using primer A1 and primer A2, introducing mutation and amplifying an antibody gene fragment B after the mutation point using primer B1 and primer B2, wherein there is an overlapping sequence between the primer B1 and the primer A2, and the primer B1 comprises a primer formed by mixing unbiased mutation primers for 18 amino acids in equal proportions, and the 18 primers are designed with mutation bases at the mutation point: the gene fragments A and B are spliced through the overlapping sequences of the primer A2 and the primer B1, and a VHH antibody gene fragment is amplified by nested PCR primers with recombination arms to obtain mutants.

Furthermore, the expression vector in the step 2) can be selected from various vectors known in the art, and then a nucleotide sequence encoding the antibody of the present invention is operably linked to an expression regulatory sequence to form an expression vector. As a preferred embodiment of the present invention, the expression vector may be pcDNA3.4, and the restriction sites are HindIII and BamHI.

Furthermore, the expression cells in the step 3) may use eukaryotic cells as host cells, preferably mammalian cells, and more preferably human embryonic kidney HEK293 cells. High-throughput expression is achieved by whole-plate expression in 96-well cell culture plates.

Furthermore, the orthogonal ELISA and sandwich ELISA method in the step 4) screen the concentration of coating human secondary antibody and antigen by the orthogonal ELISA, and the screening conditions are the concentration of coating human secondary antibody and antigen corresponding to the signal value of about 0.3. When there is more than one signal value of about 0.3, the lowest value of human secondary antibody concentration is selected. The sandwich ELISA is used to screen mutation hotspots with signal values significantly higher than the signal value of the parent.

Furthermore, the hotspot combination method in the step 5) is error-prone PCR, and if the antibody is a full-length antibody, it is constructed in the form of scFv. The screening criteria are mutation hotspot combinations with signal values significantly higher than the signal value of the parent.

Furthermore, the expression cells in the step 7) may use eukaryotic cells as host cells, preferably mammalian cells, and more preferably human embryonic kidney HEK293 cells.

In a second aspect of the present invention, a high-affinity anti-human PD-L1 single domain antibody is provided, wherein the sequence of the single domain antibody comprises a complementary determining region (CDR); the complementary determining region (CDR) comprises the amino acid sequences of CDR1, CDR2 and CDR3; the amino acid sequence of the complementary determining region (CDR) of the single domain antibody is any one of the following (1)-(15):
(1) CDR1 with an amino acid sequence of GQE, CDR2 with an amino acid sequence as set forth in SEQ ID NO: 1, and CDR3 with an amino acid sequence as set forth in SEQ ID NO: 2;
(2) CDR1 with an amino acid sequence of GME, CDR2 with an amino acid sequence as set forth in SEQ ID NO: 4, and CDR3 with an amino acid sequence as set forth in SEQ ID NO: 5;
(3) CDR1 with an amino acid sequence of GME, CDR2 with an amino acid sequence as set forth in SEQ ID NO: 7, and CDR3 with an amino acid sequence as set forth in SEQ ID NO: 8;
(4) CDR1 with an amino acid sequence of GME, CDR2 with an amino acid sequence as set forth in SEQ ID NO: 10, and CDR3 with an amino acid sequence as set forth in SEQ ID NO: 11;
(5) CDR1 with an amino acid sequence of GME, CDR2 with an amino acid sequence as set forth in SEQ ID NO: 13, and CDR3 with an amino acid sequence as set forth in SEQ ID NO: 14;
(6) CDR1 with an amino acid sequence of GME, CDR2 with an amino acid sequence as set forth in SEQ ID NO: 16, and CDR3 with an amino acid sequence as set forth in SEQ ID NO: 17;
(7) CDR1 with an amino acid sequence of GME, CDR2 with an amino acid sequence as set forth in SEQ ID NO: 19, and CDR3 with an amino acid sequence as set forth in SEQ ID NO: 20;
(8) CDR1 with an amino acid sequence of GME, CDR2 with an amino acid sequence as set forth in SEQ ID NO: 22, and CDR3 with an amino acid sequence as set forth in SEQ ID NO: 23;
(9) CDR1 with an amino acid sequence of GME, CDR2 with an amino acid sequence as set forth in SEQ ID NO: 25, and CDR3 with an amino acid sequence as set forth in SEQ ID NO: 26;
(10) CDR1 with an amino acid sequence of GME, CDR2 with an amino acid sequence as set forth in SEQ ID NO: 28, and CDR3 with an amino acid sequence as set forth in SEQ ID NO: 29;
(11) CDR1 with an amino acid sequence of GME, CDR2 with an amino acid sequence as set forth in SEQ ID NO: 31, and CDR3 with an amino acid sequence as set forth in SEQ ID NO: 32;
(12) CDR1 with an amino acid sequence of GQE, CDR2 with an amino acid sequence as set forth in SEQ ID NO: 34, and CDR3 with an amino acid sequence as set forth in SEQ ID NO: 35;
(13) CDR1 with an amino acid sequence of GQE, CDR2 with an amino acid sequence as set forth in SEQ ID NO: 37, and CDR3 with an amino acid sequence as set forth in SEQ ID NO: 38;
(14) CDR1 with an amino acid sequence of GQE, CDR2 with an amino acid sequence as set forth in SEQ ID NO: 40, and CDR3 with an amino acid sequence as set forth in SEQ ID NO: 41; or
(15) CDR1 with an amino acid sequence of GQE, CDR2 with an amino acid sequence as set forth in SEQ ID NO: 43, and CDR3 with an amino acid sequence as set forth in SEQ ID NO: 44.

Preferably, the amino acid sequence of the complementarity determining region (CDR) of the single domain antibody is selected from the above group (12) or group (15):
(12) CDR1 with an amino acid sequence of GQE, CDR2 with an amino acid sequence as set forth in SEQ ID NO: 34, and CDR3 with an amino acid sequence as set forth in SEQ ID NO: 35; or
(15) CDR1 with an amino acid sequence of GQE, CDR2 with an amino acid sequence as set forth in SEQ ID NO: 43, and CDR3 with an amino acid sequence as set forth in SEQ ID NO: 44.

Preferably, all the amino acid sequences of the CDRs above can be replaced by an amino acid sequence having at least 85% sequence homology with any one of the amino acid sequences of the CDRs of the present invention, or an amino acid sequence in which one or more amino acids in any one of the amino acid sequences of the CDRs are added, deleted or replaced.

Furthermore, the amino acid sequence of the single domain antibody is as set forth in SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42 or SEQ ID NO: 45.

Preferably, the amino acid sequence of the single domain antibody is as set forth in SEQ ID NO: 36 or SEQ ID NO: 45.

In a third aspect of the present invention, a nucleic acid is provided, wherein the nucleic acid encodes the antibody as described in the second aspect.

In a fourth aspect of the present invention, a recombinant vector is provided, wherein the recombinant vector comprises the nucleic acid as described in the third aspect.

Various vectors known in the art can be selected. For example, a commercially available vector can be selected and then a nucleotide sequence encoding the antibody of the present invention can be operably linked to an expression regulatory sequence to form an expression vector.

As a preferred embodiment of the present invention, the expression vector is pcDNA3.4.

In a fifth aspect of the present invention, a transformant is provided, wherein the transformant comprises the nucleic acid as described in the third aspect and/or the vector as described in the fourth aspect, and is capable of expressing the antibody as described in the second aspect.

Preferably, the transformant can use eukaryotic cells or prokaryotic cells as host cells, preferably mammalian cells, and more preferably human embryonic kidney HEK293 cells.

In a sixth aspect of the present invention, a pharmaceutical composition is provided, comprising the antibody as described in the second aspect, and/or the nucleic acid as described in the third aspect, and/or the vector as described in the fourth aspect, and/or the transformant as described in the fifth aspect.

Preferably, the pharmaceutical composition may further comprise pharmaceutically acceptable auxiliary materials, which may be one or more of a pharmaceutically acceptable carrier, a buffer, an excipient, a stabilizer, a preservative or other biologically active substances.

In a seventh aspect of the present invention, provided is the use of the antibody as described in the second aspect, and/or the nucleic acid as described in the third aspect, and/or the vector as described in the fourth aspect, and/or the transformant as described in the fifth aspect, and/or the composition as described in the sixth aspect as a tumor immune checkpoint inhibitor in the preparation of drugs for treating or alleviating tumors. The tumor may be melanoma or hepatoma. PD1-PDL1 immunotherapy is a broad-spectrum anti-tumor approach that can treat multiple types of tumor diseases, thereby effectively improving the overall survival of patients.

Unless otherwise defined, all technical and scientific terms used herein should be construed to have the same meanings as understood by one of ordinary skill in the art.

The definitions of the terms used in this invention are as follows:

The term “antibody” refers to a class of immunoglobulins that can specifically bind to an antigen.

The term “heavy chain” refers to the larger molecular weight peptide chain in immunoglobulin containing 440 amino acids.

The term “variable region” refers to the region of the immunoglobulin light chain and heavy chain near the N-terminus with a large variation in amino acid sequence, which is called the variable region.

The term “complementarity determining region” refers to the hypervariable regions within the variable regions of the heavy and light chains of an antibody that constitute the antigen binding sites of the antibody molecule. Because the antigen binding sites are structurally complementary to the antigen epitopes, the hypervariable regions are also called the complementarity determining regions of the antibody molecule.

The term “framework region” refers to the region other than the complementarity determining region where the composition and arrangement of amino acids are relatively unchangeable, which is called the framework region.

The term “single domain antibody (sdAb)”, also known as nanoantibody, has only one heavy chain variable region domain (VHH). This domain was originally found in an antibody HCAb isolated from the serum of camelids and sharks, and the VHH fragment was amplified through genetic means. The VHH region cloned and expressed separately has good structural stability and antigen binding activity. VHH is the smallest unit known to bind to the target antigen.

The term “affinity maturation” refers to an in vitro affinity maturation technology of antibodies, which mainly simulates the in vivo affinity maturation process. Various strategies are used for respective mutations of antibody genes, a mutant antibody library is constructed, and high-affinity antibodies are obtained through affinity screening.

The present invention has the following technical effects:
1) The affinity maturation method of the present invention has the technical effect of improving antibody affinity, and this effect has been verified in Example 7.
2) The anti-human PD-L1 single domain antibody of the present invention has greatly improved affinity, by up to 10 times or more, compared with the parent antibody, and the affinity-matured antibody still has the activity of blocking the binding of PD-L1 to PD-1, and this effect has been verified in Examples 7 and 9.

### BRIEF DESCRIPTION OF THE DRAWINGS

FIG. 1 shows the PCR process in Example 1.

FIG. 2 shows a map of B378737 in Example 1.

FIG. 3 shows a map of pcDNA3.4 in Example 1.

FIG. 4 shows an agarose gel electrophoresis diagram of the plasmid in Example 2.

FIG. 5 shows some hotspot screening results of Example 4.

FIG. 6 shows the relative affinity ranking results by ELISA of Example 6. Both a and b are the relative affinity ranking results by ELISA of the affinity matured antibodies, which are the results of two-plate ELISA.

FIG. 7 shows the SDS-PAGE result under reducing conditions of Example 7.

FIG. 8 shows the affinity test result by Biocore 8K of Example 7. Herein, a is the binding kinetic curve of the parent antibody B378737, b is the binding kinetic curve of the affinity matured antibody B378737-ZH-1, and c is the binding kinetic curve of the affinity matured antibody B378737-ZH-4.

FIG. 9 shows a summary of the affinity maturation process of Example 8.

FIG. 10 shows the validation of the blocking activity of affinity matured antibodies.

### DESCRIPTION OF THE EMBODIMENTS

In order to make the objectives, technical solutions, and advantages of the present invention clearer, the present invention is further described in detail below with reference to embodiments and accompanying drawings. Obviously, the described embodiments are only part of the embodiments of the present invention, rather than all the embodiments.

Taking the affinity maturation of anti-human PD-L1 single domain antibody as an example, the technology of the present invention is described in detail.

The screening process of anti-human PD-L1 single domain antibody (B378737) is as follows: After immunizing alpaca (Vicugna pacos) with human PD-L1, alpaca PBMC cells are extracted to obtain antibody gene fragments, and anti-human PD-L1 single domain antibody (B378737) is screened based on phage display technology. Its nucleotide sequence is as set forth in SEQ ID NO: 69, and its amino acid sequence is as set forth in SEQ ID NO: 70.

### Example 1 Construction and Verification of Mutants by Unbiased, Full-Coverage Single-Point Saturation Mutation

The FR and CDR regions of antibody B378737 were annotated according to the Kabat scheme. The nucleotide sequence is as set forth in SEQ ID NO: 69, and the amino acid sequence is as set forth in SEQ ID NO: 70. There are 26 amino acids in total in the three CDRs. The process of unbiased full-coverage single-point saturation mutation is introduced by taking the mutation of the first amino acid of CDR1, glycine (G), to the other 18 amino acids as an example.

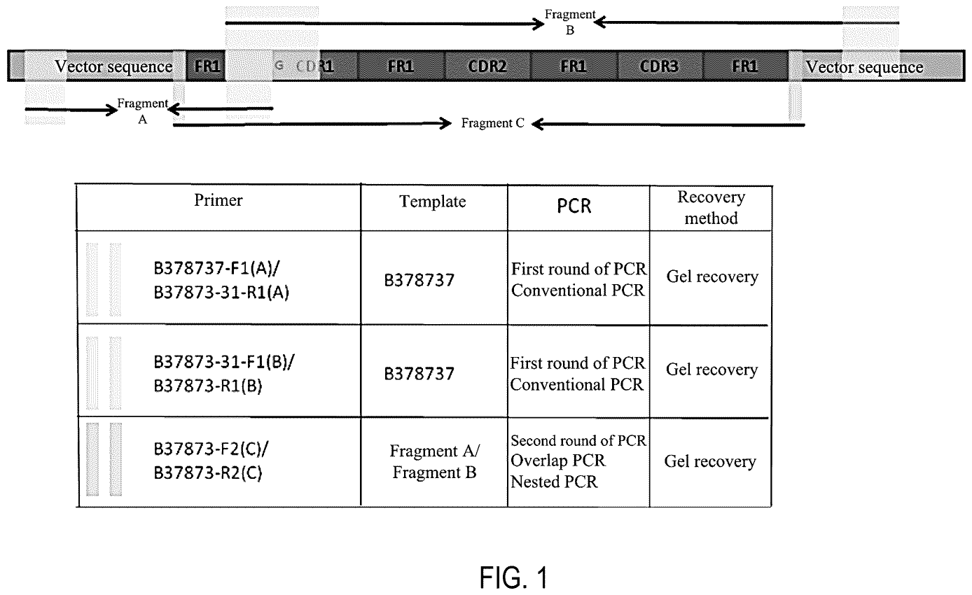

FIG. 1 shows the PCR idea and process of a full mutation of DNA, which is briefly summarized as follows: a fragment A before a mutation point is amplified by primers B378737-F1(A)/B37873-31-R1(A), and mutation is introduced and a fragment B after the mutation point is amplified by primers B37873-31-F1(B)/B37873-R1 (B), wherein B37873-31-F1(B) is a primer mixed in equal proportions of 18 primers, and these 18 primers are designed with mutation bases at mutation point G, the fragments A and B are spliced through the overlapping sequences of primers B37873-31-R1(A) and B37873-31-F1(B), and a VHH antibody fragment C is amplified by nested PCR primers B37873-F2(C)/B37873-R2(C) with recombination arms. The PCR primer sequences are shown in Table 2.

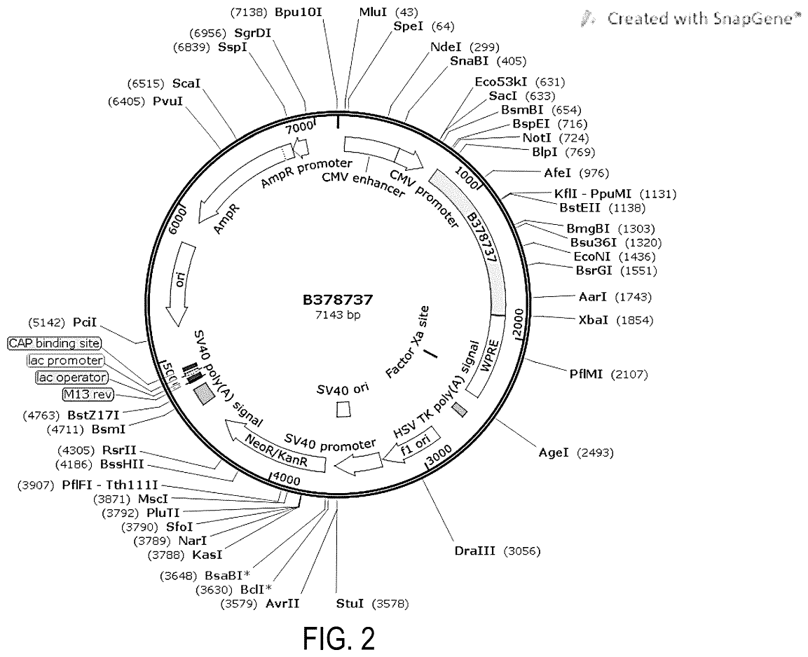

The plasmid map of B378737 is shown in FIG. 2, which is obtained by connecting the coding gene of the B378737 antibody with the vector pcDNA3.4, and the restriction site is not1/xba1.

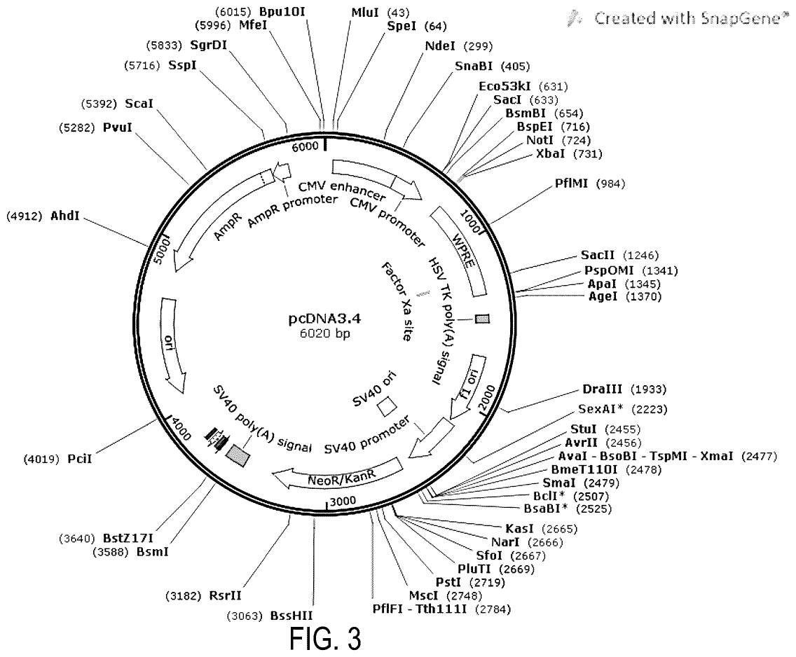

The nested PCR product was recovered by gel cutting and then recombined with the vector pcDNA3.4 (HindIII/BamHI). The map of pcDNA3.4 is shown in FIG. 3.

The recombinant was transferred into TOP10 competent cells by heat shock method, spread on ampicillin-resistant plates, and cultured at 37° C. overnight. The mutation construction method of the other 25 amino acids was the same as above.

88 monoclones constructed after randomly selecting one amino acid (such as 31G) for mutation were sent for sequencing. The analysis of sequencing results is shown in Table 3. The sequencing results show that the 88 clones sent for sequencing contain all 18 designed amino acids, the mutation coverage rate reachs 100%, and the positive rate of insertion is 90%, indicating successful mutation.

TABLE 2
PCR primer sequences

| SEQ ID | | |
|---|---|---|
| NO | Name | 5′_3′ |
| SEQ ID | B378737-F1(A) | CAACTCCGCCCCATTGACGCAAAT |
| NO: 46 | | |
| SEQ ID | B37873-31-R1(A) | AAATGTAAACCCGCTGGCGGCACAG |
| NO: 47 | | |
| SEQ ID | B37873-31-F1(B) | CTGTGCCGCCAGCGGGTTTACATTTGCCT |
| NOs: | 18 primers; | TTAGGCACTATGTGATGGG |
| 48-65 | mixed in equal | CTGTGCCGCCAGCGGGTTTACATTTGACT |
| | proportions | TTAGGCACTATGTGATGGG |
| | | CTGTGCCGCCAGCGGGTTTACATTTGAGT |
| | | TTAGGCACTATGTGATGGG |
| | | CTGTGCCGCCAGCGGGTTTACATTTTTCT |
| | | TTAGGCACTATGTGATGGG |
| | | CTGTGCCGCCAGCGGGTTTACATTTGGCT |
| | | TTAGGCACTATGTGATGGG |
| | | CTGTGCCGCCAGCGGGTTTACATTTCACT |
| | | TTAGGCACTATGTGATGGG |
| | | CTGTGCCGCCAGCGGGTTTACATTTATCT |
| | | TTAGGCACTATGTGATGGG |
| | | CTGTGCCGCCAGCGGGTTTACATTTAAGT |
| | | TTAGGCACTATGTGATGGG |
| | | CTGTGCCGCCAGCGGGTTTACATTTCTGT |
| | | TTAGGCACTATGTGATGGG |
| | | CTGTGCCGCCAGCGGGTTTACATTTATGT |
| | | TTAGGCACTATGTGATGGG |
| | | CTGTGCCGCCAGCGGGTTTACATTTAACT |
| | | TTAGGCACTATGTGATGGG |
| | | CTGTGCCGCCAGCGGGTTTACATTTCCCT |
| | | TTAGGCACTATGTGATGGG |
| | | CTGTGCCGCCAGCGGGTTTACATTTCAGT |
| | | TTAGGCACTATGTGATGGG |
| | | CTGTGCCGCCAGCGGGTTTACATTTCGCT |
| | | TTAGGCACTATGTGATGGG |
| | | CTGTGCCGCCAGCGGGTTTACATTTTCCT |
| | | TTAGGCACTATGTGATGGG |
| | | CTGTGCCGCCAGCGGGTTTACATTTGTGT |
| | | TTAGGCACTATGTGATGGG |
| | | CTGTGCCGCCAGCGGGTTTACATTTTGGT |
| | | TTAGGCACTATGTGATGGG |
| | | CTGTGCCGCCAGCGGGTTTACATTTTACT |
| | | TTAGGCACTATGTGATGGG |
| SEQ ID | B37873-R1(B) | ACACGGTACGTGCTGTTGTACTGC |
| NO: 66 | | |
| SEQ ID | B37873-F2(C) | GTCCTCCTGACTGGGGTGAGGGCC |
| NO: 67 | | |
| SEQ ID | B37873-R2(C) | AGTTTTGTCAGCAGATTTGGGCTC |
| NO: 68 | | |

TABLE 3
31G amino acid mutation results

| | | |
|---|---|---|
| | Number of | Frequency of |
| Amino acids | occurrences | occurrences |

| | | | |
|---|---|---|---|
| Alanine | A | 2 | 2.27% |
| Arginine | R | 3 | 3.41% |
| Asparagine | N | 3 | 3.41% |
| Aspartic acid | D | 7 | 7.95% |
| Glutamine | Q | 3 | 3.41% |
| Glutamate | E | 4 | 4.55% |
| Histidine | H | 5 | 5.68% |
| Isoleucine | I | 5 | 5.68% |
| Lysine | K | 3 | 3.41% |
| Methionine | M | 5 | 5.68% |
| Phenylalanine | F | 6 | 6.82% |
| Proline | P | 3 | 3.41% |
| Serine | S | 4 | 4.55% |
| Threonine | T | 4 | 4.55% |
| Tryptophan | W | 6 | 6.82% |
| Tyrosine | Y | 6 | 6.82% |
| Valine | V | 4 | 4.55% |
| Leucine | L | 6 | 6.82% |
| Glycine | G | 0 | 0.00% |
| Cysteine | C | 0 | 0.00% |

| | | |
|---|---|---|
| Insert error sequence | 9 | 10.23% |
| Total number of clones | 88 | 100.00% |

### Example 2 High-Throughput Plasmid Extraction

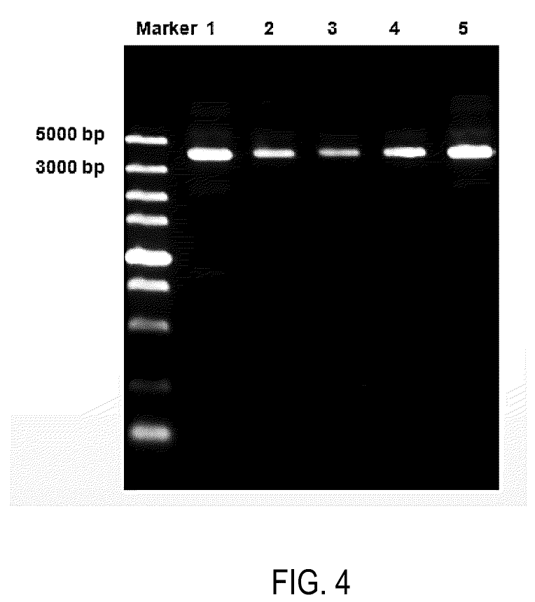

After mutating the 26 amino acids in the CDR region according to the above scheme, a total of 26 transformation plates were obtained. 88 monoclones were randomly selected from each plate and placed in columns 1-11 of a 96-deep-well plate. In 12A-12D, the monoclones of parent B378737 were selected as positive controls, 12E-12F were used as negative controls in subsequent experiments, and 12G-12H were used as blank controls in subsequent experiments. Then a whole plate of B378737 monoclonal clones was selected for subsequent orthogonal ELISA. It was cultured overnight at 220 rpm with 600 μL of ampicillin-resistant 2×YT medium, and the plasmid was extracted by alkaline lysis and alcohol precipitation the next day. The specific process is as follows: it was centrifuged at 4000 r for 5 min and the supernatant was discarded; suspension S1 with RNase A was added to each well and shaken evenly; lysis solution S2 was added to each well to clarify the bacterial solution; neutralization solution S3 was added to each well, shaken and mixed evenly, and it was centrifuged at 4000 rpm for 10 min; the supernatant of each well was filtered and mixed evenly with isopropanol, it was centrifuged at 4000 r for 10 min, and then the supernatant was discarded; 70% ethanol was added to each well for washing, and it was centrifuged at 4000 r for 5 min, and the supernatant was discarded; the remainer was air-dried for 3-5 min to evaporate the ethanol; 150 μL of deionized water was added to each well to dissolve the plasmid; and the plasmids from 5 wells were randomly selected to measure the concentration and subjected to agarose gel electrophoresis. The results are shown in FIG. 4, which show that the plasmid bands are complete and of correct size, and the plasmid extraction is successful.

### Example 3 High-Throughput Expression

The following reagent volumes are for one well of a 96-well cell culture plate. 10 μL (about 500 ng) plasmid was diluted with 15 μL Hybridoma medium, and the transfection reagent was diluted with Hybridoma medium. The diluted transfection reagent was added to the diluted plasmid, and after 15 minutes, 200 μL of HEK293 cells passaged to the third generation were added. Hybridoma and DMEM were mixed in equal proportions, and they were cultured in 1.2% FBS, at 37° C., 5% CO₂ for 96 h. The expression supernatants from 5 wells were randomly selected for concentration measurement, and the results are shown in Table 4. The results show that the average antibody expression level is 10 μg/mL, and the antibody expression is successful.

TABLE 4
Results of antibody concentration test in expression
supernatant

| Sample name | Concentration μg/mL |
|---|---|
| B378737-31G-1 | 12.9 |
| B378737-31G-8 | 8.2 |
| B378737-31G-37 | 9.2 |
| B378737-31G-81 | 8.5 |
| B378737-31G-88 | 9.3 |

### Example 4 Screening of Mutation Hotspots

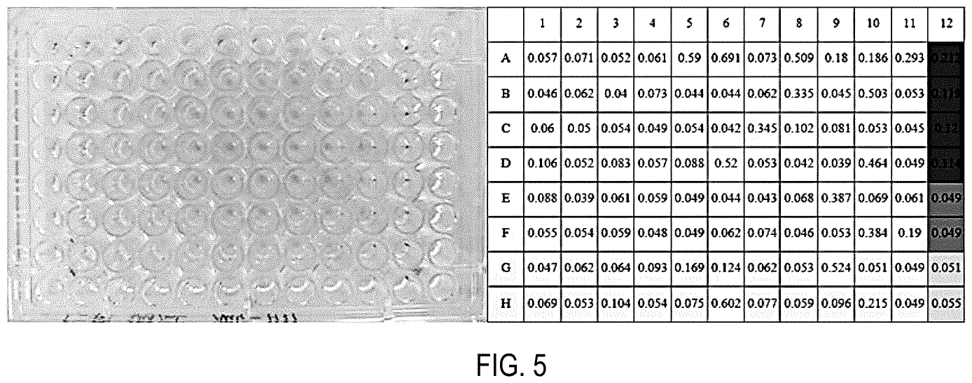

Goat-anti-human IgG Fc was subjected to serial dilution in Table 5 and coating was performed overnight at 4° C. The coating solution was discarded the next day, and the plate was washed 5 times. Blocking was performed with 1% BSA at room temperature for 1 h, and the plate was washed 5 times. 50 μL B378737 expression supernatant was added to each well, incubation was performed at room temperature for 1 h, and the plate was washed 5 times. Biotin-labeled PDL1/His protein diluted in series was added, incubation was performed at room temperature for 1 h, and the plate was washed 5 times. SA-HRP was added, incubation was performed at room temperature for 0.5 h, and the plate was washed 10 times. TMB color development was terminated after 4 min, and the absorbance at 450 nm was read. The results are shown in Table 5. The concentration of Goat-anti-human IgG Fc at 1.0 μg/mL and the concentration of Bio-PDL1 at 4 ng/ml were selected as the concentrations used for subsequent hotspot screening. The sample was the mutant expressing supernatant, and the other processes were the same as above. The mutant plasmids with significantly higher signal values than that of the parent were screened out and sent for sequencing. Some hotspot screening results are shown in FIG. 5. Table 7 shows the sequencing results of hotspots and hotspot combinations, where the underlined ones are the mutation positions.

TABLE 5
Orthogonal ELISA process and results

| | | [Anti-human Fc Ab] | | | | | |
|---|---|---|---|---|---|---|---|
| | | 2 | 1.5 | 1.0 | 0.75 | 0.5 | 0.25 |
| [Bio-PDL1] | | ug/ml | ug/ml | ug/ml | ug/ml | ug/ml | ug/ml |
| 500 | ng/ml | 2.413 | 2.493 | 2.398 | 2.659 | 2.168 | 1.61 |
| 250 | ng/ml | 2.186 | 2.422 | 2.344 | 2.366 | 1.769 | 1.462 |
| 125 | ng/ml | 2.27 | 2.296 | 2.145 | 2.118 | 1.852 | 1.304 |
| 62.5 | ng/ml | 2.058 | 2.014 | 2.018 | 1.73 | 1.322 | 0.782 |
| 31.25 | ng/ml | 1.569 | 1.639 | 1.523 | 1.452 | 0.862 | 0.417 |
| 15.625 | ng/ml | 1.502 | 1.192 | 1.076 | 0.915 | 0.377 | 0.181 |
| 7.8125 | ng/ml | 0.859 | 0.702 | 0.489 | 0.422 | 0.19 | 0.086 |
| 3.90625 | ng/ml | 0.384 | 0.385 | 0.252 | 0.219 | 0.093 | 0.072 |
| 2.109375 | ng/ml | 0.226 | 0.156 | 0.119 | 0.111 | 0.077 | 0.076 |
| 1.0546875 | ng/ml | 0.098 | 0.079 | 0.075 | 0.066 | 0.056 | 0.065 |
| 0.52734375 | ng/ml | 0.073 | 0.066 | 0.057 | 0.056 | 0.051 | 0.058 |
| 0.263671875 | ng/ml | 0.062 | 0.054 | 0.051 | 0.05 | 0.048 | 0.09 |
| 0.1318359375 | ng/ml | 0.051 | 0.049 | 0.048 | 0.047 | 0.048 | 0.056 |
| 0.06591796875 | ng/ml | 0.048 | 0.046 | 0.047 | 0.047 | 0.048 | 0.054 |
| 0.032958984375 | ng/ml | 0.048 | 0.109 | 0.046 | 0.047 | 0.049 | 0.044 |
| 0 | ng/ml | 0.051 | 0.051 | 0.052 | 0.05 | 0.059 | 0.046 |

### Example 5 Mutation Hotspot Combinations

The 11 mutation molecular plasmids screened out in Example 4 were mixed in equal proportions, and 20 ng of the mixed plasmid was taken as a template to perform error-prone PCR for hotspot combinations according to the process in Table 6. After gel recovery, the recombinant vector was constructed. The next day, 10 plates of 96-well monoclones were selected, and the subsequent operations were carried out according to Examples 2-4. The plasmids with hotspot combinations that had significantly higher signal values than that of the parent were sent for sequencing. The sequencing results of hotspot combinations are shown in Table 7.

TABLE 6
Error-prone PCR system and procedures

| mutation plasmids | 20 | ng |
|---|---|---|
| 10 μM B37873-F2(C) | 1 | μL |
| 10 μM B37873-R2(C) | 1 | μL |
| 5X PCR buffer | 10 | μL |
| dNTP mix | 1 | μL |
| Taq polymerase | 1 | μL |
| ddH20 | up to 50 μL | |
| 94° C., 30 s | 80 | cycles |
| 55° C., 5 s | | |

TABLE 7
Sequencing results of hotspots and hotspot combinations

| Clone | Variable region amino acid | | | |
|---|---|---|---|---|
| number | sequence of single-chain antibody | CDR1 | CDR2 | CDR3 |
| B378737- | QVQLVESGGGLVQPGGSLRLSCAASARSING | GQE | GITAG | QYG |
| 32 | QEWYRQAPGERRELVAGITAGGSAYYTDTVK | | GSAYY | PNW |
| | GRFTISRDNAENTGYLQMNSLSPDDTAVYYC | | TDTVK | Y |
| | RRQYGPNWYWGQGTQVTVSS | | G | |
| B378737- | QVQLVESGGGLVQPGGSLRLSCAASARSING | GME | GITSGG | QYG |
| 51 | MEWYRQAPGERRELVAGITSGGSAYYTDTVK | | SAYYT | PNW |
| | GRFTISRDNAENTGYLQMNSLSPDDTAVYYC | | DTVKG | Y |
| | RRQYGPNWYWGQGTQVTVSS | | | |
| B378737- | QVQLVESGGGLVQPGGSLRLSCAASARSING | GME | GITAG | QYG |
| 61-1 | MEWYRQAPGERRELVAGITAGGSAYYTDTPK | | GSAYY | PNW |
| | GRFTISRDNAENTGYLQMNSLSPDDTAVYYC | | TDTPK | Y |
| | RRQYGPNWYWGQGTQVTVSS | | G | |
| B378737- | QVQLVESGGGLVQPGGSLRLSCAASARSING | GME | GITAG | QYG |
| 61-2 | MEWYRQAPGERRELVAGITAGGSAYYTDTY | | GSAYY | PNW |
| | KGRFTISRDNAENTGYLQMNSLSPDDTAVYY | | TDTYK | Y |
| | CRRQYGPNWYWGQGTQVTVSS | | G | |
| B378737- | QVQLVESGGGLVQPGGSLRLSCAASARSING | GME | GITAG | QYG |
| 61-3 | MEWYRQAPGERRELVAGITAGGSAYYTDTSK | | GSAYY | PNW |
| | GRFTISRDNAENTGYLQMNSLSPDDTAVYYC | | TDTSK | Y |
| | RRQYGPNWYWGQGTQVTVSS | | G | |
| B378737- | QVQLVESGGGLVQPGGSLRLSCAASARSING | GME | GITAG | QYG |
| 61-4 | MEWYRQAPGERRELVAGITAGGSAYYTDTT | | GSAYY | PNW |
| | KGRFTISRDNAENTGYLQMNSLSPDDTAVYY | | TDTTK | Y |
| | CRRQYGPNWYWGQGTQVTVSS | | G | |
| B378737- | QVQLVESGGGLVQPGGSLRLSCAASARSING | GME | GITAG | QYG |
| 61-5 | MEWYRQAPGERRELVAGITAGGSAYYTDTH | | GSAYY | PNW |
| | KGRFTISRDNAENTGYLQMNSLSPDDTAVYY | | TDTHK | Y |
| | CRRQYGPNWYWGQGTQVTVSS | | TT | |
| | | | G | |
| B378737- | QVQLVESGGGLVQPGGSLRLSCAASARSING | GME | GITAG | QYG |
| 61-6 | MEWYRQAPGERRELVAGITAGGSAYYTDTK | | GSAYY | PNW |
| | KGRFTISRDNAENTGYLQMNSLSPDDTAVYY | | TDTKK | Y |
| | CRRQYGPNWYWGQGTQVTVSS | | G | |
| B378737- | QVQLVESGGGLVQPGGSLRLSCAASARSING | GME | GITAG | QYG |
| 61-7 | MEWYRQAPGERRELVAGITAGGSAYYTDTL | | GSAYY | PNW |
| | KGRFTISRDNAENTGYLQMNSLSPDDTAVYY | | TDTLK | Y |
| | CRRQYGPNWYWGQGTQVTVSS | | G | |
| B378737- | QVQLVESGGGLVQPGGSLRLSCAASARSING | GME | GITAG | QFGP |
| 97-1 | MEWYRQAPGERRELVAGITAGGSAYYTDTV | | GSAYY | NWY |
| | KGRFTISRDNAENTGYLQMNSLSPDDTAVYY | | TDTVK | |
| | CRRQFGPNWYWGQGTQVTVSS | | G | |
| B378737- | QVRLVESGGGLVQPGGSLRLSCAASARSING | GME | GITAG | QVG |
| 97-2 | MEWYRQAPGERRELVAGITAGGSAYYTDTV | | GSAYY | PNW |
| | KGRFTISRDNAENTGYLQMNSLSPDDTAVYY | | TDTVK | Y |
| | CRRQVGPNWYWGQGTQVTVSS | | G | |
| B378737- | QVQLVESGGGLVQPGGSLRLSCAASARSING | GQE | GITAG | QFGP |
| ZH-1 | QEWYRQAPGERRELVAGITAGGSAYYTDTVK | | GSAYY | NWY |
| | GRFTISRDNAENTGYLQMNSLSPDDTAVYYC | | TDTVK | |
| | RRQFGPNWYWGQGTQVTVSS | | G | |
| B378737- | QVQLVESGGGLVQPGGSLRLSCAASARSING | GQE | GITAG | QVG |
| ZH-2 | QEWYRQAPGERRELVAGITAGGSAYYTDTVK | | GSAYY | PNW |
| | GRFTISRDNAENTGYLQMNSLSPDDTAVYYC | | TDTVK | Y |
| | RRQVGPNWYWGQGTQVTVSS | | G | |
| B378737- | QVQLVESGGGLVQPGGSLRLSCAASARSING | GQE | GITAGS | QFGP |
| ZH-3 | QEWYRQAPGERRELVAGITAGGSAYYTDTHK | | AYYTD | NWY |
| | GRFTISRDNAENTGYLQMNSLSPDDTAVYYC | | THKG | |
| | RRQEGPNWYWGQGTQVTVSS | | | |
| B378737- | QVQLVESGGGLVQPGGSLRLSCAASARSING | GQE | GITAGS | QVG |
| ZH-4 | QEWYRQAPGERRELVAGITAGGSAYYTDTHK | | AYYTD | PNW |
| | GRFTISRDNAENTGYLQMNSLSPDDTAVYYC | | THKG | Y |
| | RRQVGPNWYWGQGTQVTVSS | | | |

In Table 7, the amino acid sequences of the variable regions are the sequencing results of all VHHs. The underlined amino acids are hotspots, and those with two or more hotspots are combinations.

### Example 6 Relative Affinity Ranking

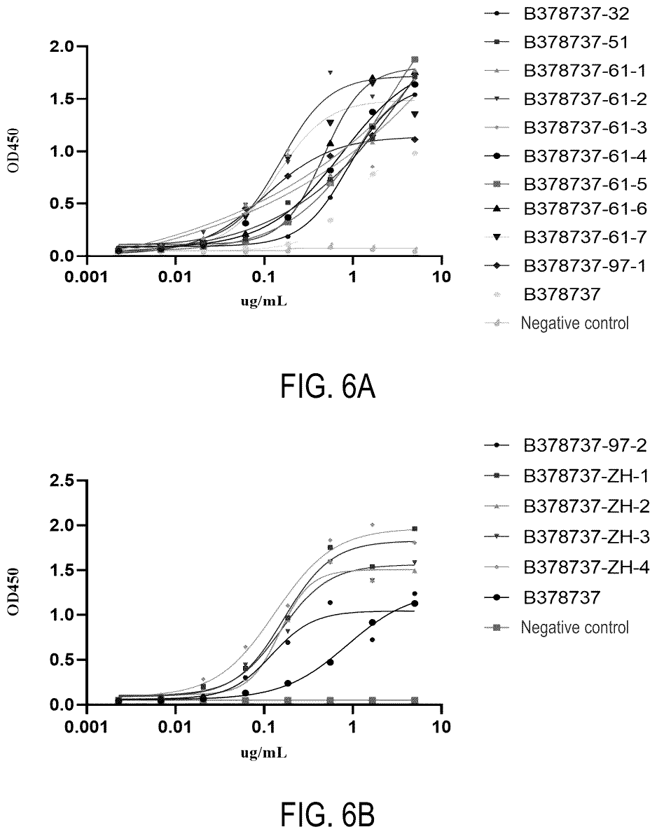

The expression supernatants of the 11 single-point mutation molecules screened out in Example 4 and the 4 hotspot combination molecules screened out in Example 5 were subjected to relative affinity ranking. The specific process is as follows: The plate was coated with 1 μg/mL Goat-anti-human IgG Fc at 4° C. overnight. The coating solution was discarded the next day, and the plate was washed 5 times. Blocking was performed with 1% BSA at room temperature for 1 h, and the plate was washed 5 times. 50 μL of 2-fold diluted expression supernatant was added to each well and incubation was performed at room temperature for 1 h, and the plate was washed 5 times. Biotin-labeled PDL1/His diluted in series was added with 5 μg/mL in the first well, 3-fold dilution, and the last well as blank, and incubation was performed at room temperature for 1 h, and the plate was washed 5 times. SA-HRP was added, and incubation was performed at room temperature for 0.5 h, and the plate was washed 10 times. TMB color development was terminated after 4 min, and the absorbance at 450 nm was read. The results are shown in a and b in FIG. 6.

It can be seen from a and b in FIG. 6 that the relative affinity ranking of both the antibody with single-point mutation and the antibody with multi-point mutation is higher than that of the parent sequence.

### Example 7 Expression, Purification and Affinity Testing of Antibody Molecules

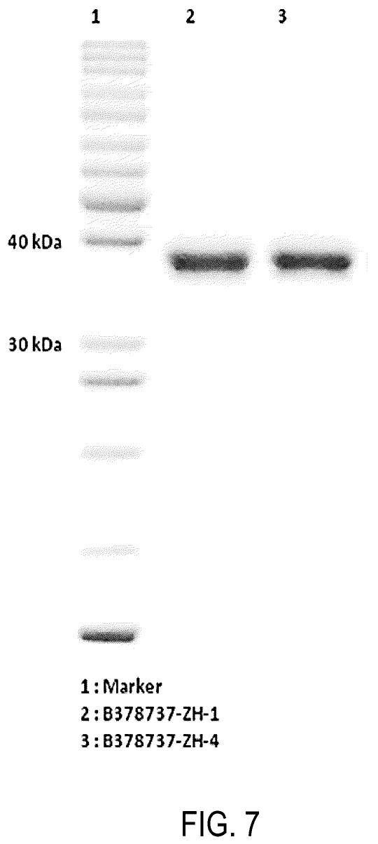

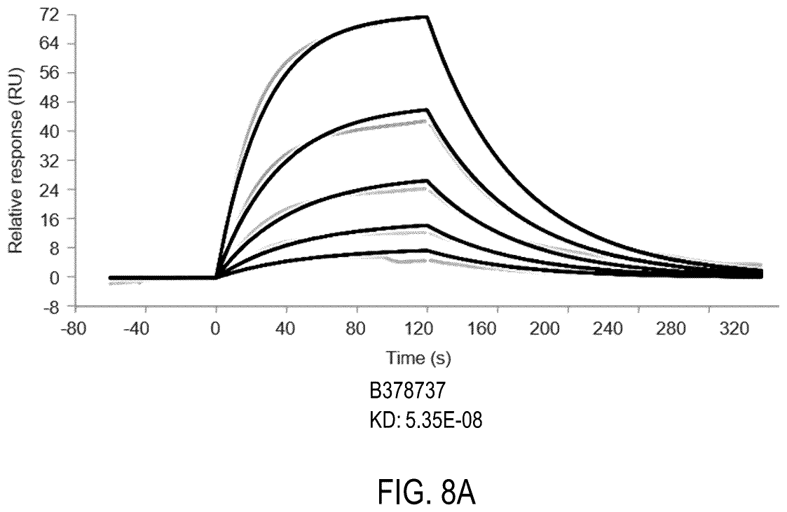

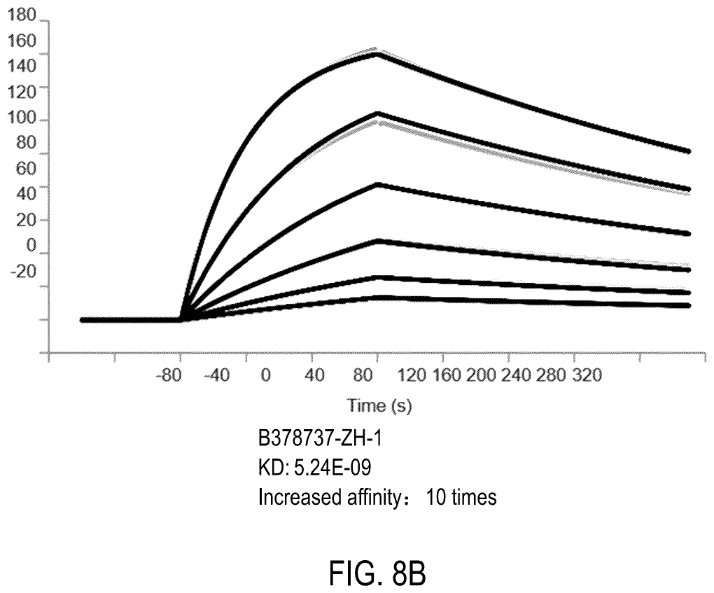

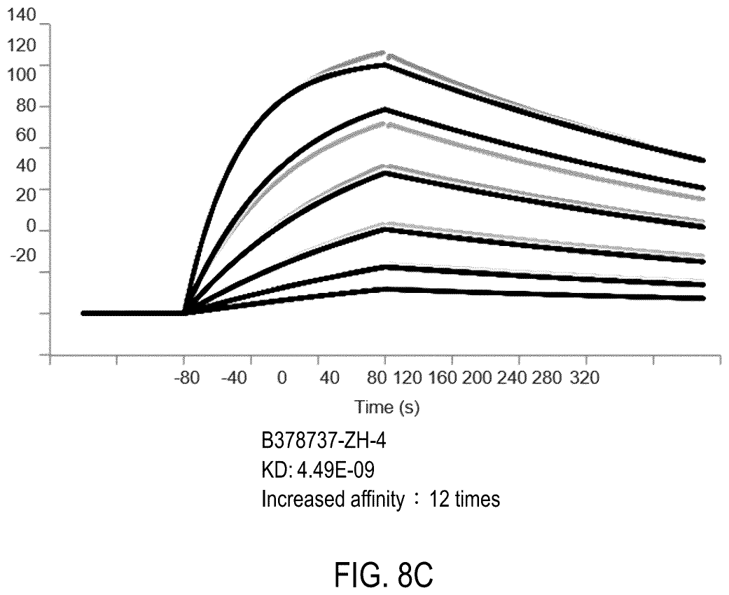

The 2 molecules B378737-ZH-1 and B378737-ZH-4 with the highest relative affinity in Example 6 were selected for small-scale expression and purification in HEK-293 cells, as follows: the successfully constructed recombinant vector was transfected into HEK-293 cells. HEK-293 cells in logarithmic growth phase were inoculated into 6-well plates at a cell density of 1.5×10⁶ cell/mL, cultured at 37° C., 5% CO₂ in an incubator at 600 rpm, and transfected 2 h later. Feed and fluid were supplemented on days 2, 4, and 6 of culture, and samples were collected and purified on day 7. The samples were collected in separate tubes, with approximately 500 μL per tube. A total of 10 tubes were collected and the absorbance at 280 nm was read using a NanoDrop instrument. The high-concentration protein was aspirated into a dialysis bag for dialyzation, and the purified antibody was collected. The SDS-PAGE results under reducing conditions are shown in FIG. 7. The test results by Biacore 8K are shown in FIG. 8. As can be seen from a, b, and c in FIG. 8, compared with the parent antibody B378737, the affinities of B378737-ZH-1 and B378737-ZH-4 are increased by 10 and 12 times, respectively, and it can be considered that affinity maturation was successful.

### Example 8 Summary of Affinity Maturation Method

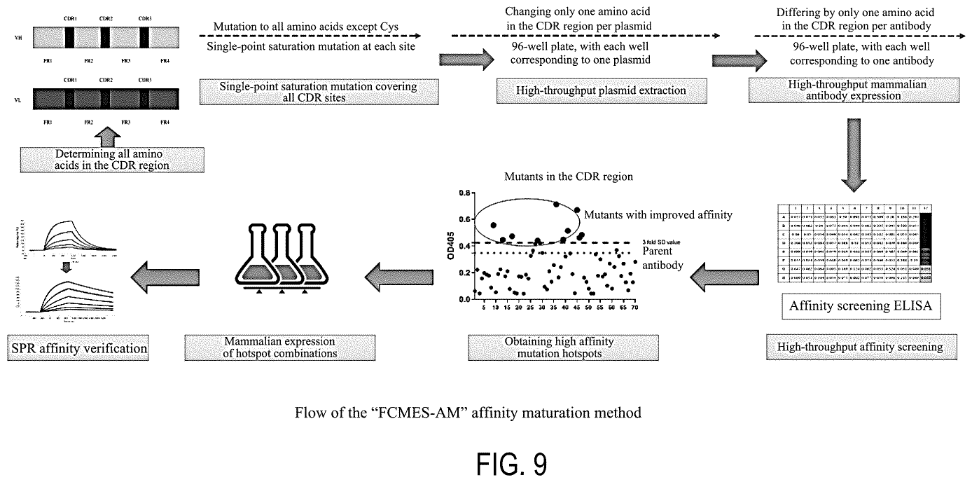

The affinity maturation process of Example 8 is shown in FIG. 9. Single-point saturation mutation of all amino acids in the CDR region was achieved by designing and synthesizing unbiased mutation primers for 18 amino acids. Mutation hotspots were screened through mammalian high-throughput expression and ELISA detection of the expression supernatant. The combination of mutation hotspots was accomplished through error-prone PCR. Finally, the affinity improvement was detected through antibody expression and purification, and biomolecular interaction analysis system. The entire process was independently developed and completed by the FC-MES affinity maturation platform of JIANGSU BIOINTRON TECH CO LTD.

### Example 9 ELISA Verification of the Blocking Activity of Affinity Matured Antibodies

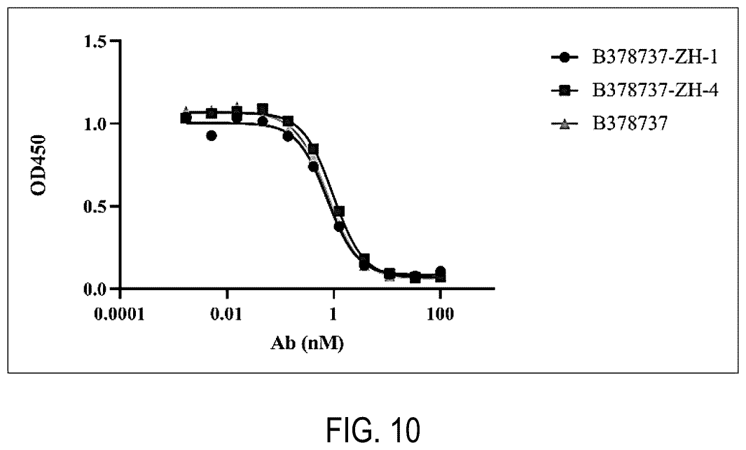

The blocking activity of the antibodies B378737-ZH-1 and B378737-ZH-4 successfully affinity-matured in Example 7 was verified by ELISA and compared with that of the parent antibody B378737. The specific process is as follows: The plate was coated with 5 μg/mL PDL1/His overnight at 4° C. The coating solution was discarded the next day, and the plate was washed 5 times. Blocking was performed with 1% BSA at room temperature for 1 h, and the plate was washed 5 times. 100 μL antibody was added to each well with 100 nM in the first well, 3-fold dilution, 12 points and the last well as blank, and incubation was performed at room temperature for 1 h, and the plate was washed 5 times. 100 μL of 10 μg/mL biotin-labeled PD1/His was added to each well, incubation was performed at room temperature for 1 h, and the plate was washed 5 times. SA-HRP was added, incubation was performed at room temperature for 0.5 h, and the plate was washed 10 times. TMB color development was terminated after 4 min, and the absorbance at 450 nm was read. The results are shown in FIG. 10. The results show that the affinity matured antibodies still have the activity of blocking the binding of PDL1 to PD1, and the blocking activity is comparable to that of the parent antibody.

The above are only embodiments of the present invention, and are not intended to limit the patent scope of the present invention. Any equivalent transformations made from the contents of the present specification, or direct or indirect applications in other related technical fields, are also included in the patent protection scope of the present invention.

1. An antibody affinity maturation method, which is a method of combining a single-point saturation mutation with full coverage of a CDR region and a mammalian ultra-high throughput expression screening system; and
a method of the single-point saturation mutation with full coverage of the CDR region is a method of unbiased and full-coverage mutation of all amino acids in the CDR region of antibodies via a mixture of equal proportions of primers.

2. The antibody affinity maturation method according to claim 1, wherein a method of the mammalian ultra-high throughput expression screening system comprises steps of:
1) simultaneously expressing thousands of microwells using 96-well cell culture plates with each well expressing only a single antibody with one amino acid mutation to cover all mutation points in the CDR region; and
2) performing an affinity screening on all expression supernatants using an affinity screening ELISA method to obtain high-affinity mutation hotspots.

3. The antibody affinity maturation method according to claim 1, wherein expression screening cells used in a method of the mammalian ultra-high throughput expression screening system are mammalian expression cells, which eliminates an influence of display system proteins on an antibody detection in conventional display systems.

4. The antibody affinity maturation method according to claim 2, wherein the affinity screening ELISA method is a method of capturing antibodies in the expression supernatants uniformly to eliminate an influence of concentration differences, thereby reflecting affinity differences through color development differences.

5. The antibody affinity maturation method according to claim 1, comprising steps of:
1) constructing and verifying mutants of an antibody by a single-point saturation mutation technique;
2) recombining the mutants into an expression vector;
3) performing a high-throughput extraction of plasmids and a transfection of mammalian cells for expression;
4) screening mutation hotspots by orthogonal ELISA and sandwich ELISA;
5) performing a hotspot combination by an error-prone PCR;
6) performing a relative affinity ranking of antibodies of the hotspot combination by sandwich ELISA; and
7) selecting antibodies with an improved affinity for expression and affinity detection.

6. The affinity maturation method according to claim 5, wherein the step 1) comprises constructing the mutants of all amino acids in CDRs of the antibody, and a process of single-point saturation mutation of each amino acid is as follows: amplifying an antibody gene fragment A before a mutation point using a primer A1 and a primer A2, introducing a mutation and amplifying an antibody gene fragment B after the mutation point using a primer B1 and a primer B2, wherein there is an overlapping sequence between the primer B1 and the primer A2, and the primer B1 comprises a primer formed by mixing unbiased mutation primers for 18 amino acids in equal proportions, and 18 primers are designed with mutation bases at the mutation point: the gene fragments A and B are spliced through the overlapping sequences of the primers A2 and B1, and a VHH antibody gene fragment is amplified by nested PCR primers with recombination arms to obtain mutants.

7. The affinity maturation method according to claim 5, wherein in the step 2), the expression vector is pcDNA3.4, and restriction sites are HindIII and BamHI.

8. The affinity maturation method according to claim 5, wherein in the step 3), expression cells are human embryonic kidney HEK293 cells.

9. A high-affinity anti-human PD-L1 single domain antibody, wherein a sequence of the high-affinity anti-human PD-L1 single domain antibody comprises a complementary determining region (CDR); the complementary determining region (CDR) comprises amino acid sequences of CDR1, CDR2 and CDR3; an amino acid sequence of the complementary determining region (CDR) of the high-affinity anti-human PD-L1 single domain antibody is any one of following (1)-(15):
(1) CDR1 with an amino acid sequence of GQE, CDR2 with an amino acid sequence as set forth in SEQ ID NO: 1, and CDR3 with an amino acid sequence as set forth in SEQ ID NO: 2;
(2) CDR1 with an amino acid sequence of GME, CDR2 with an amino acid sequence as set forth in SEQ ID NO: 4, and CDR3 with an amino acid sequence as set forth in SEQ ID NO: 5;
(3) CDR1 with an amino acid sequence of GME, CDR2 with an amino acid sequence as set forth in SEQ ID NO: 7, and CDR3 with an amino acid sequence as set forth in SEQ ID NO: 8;
(4) CDR1 with an amino acid sequence of GME, CDR2 with an amino acid sequence as set forth in SEQ ID NO: 10, and CDR3 with an amino acid sequence as set forth in SEQ ID NO: 11;
(5) CDR1 with an amino acid sequence of GME, CDR2 with an amino acid sequence as set forth in SEQ ID NO: 13, and CDR3 with an amino acid sequence as set forth in SEQ ID NO: 14;
(6) CDR1 with an amino acid sequence of GME, CDR2 with an amino acid sequence as set forth in SEQ ID NO: 16, and CDR3 with an amino acid sequence as set forth in SEQ ID NO: 17;
(7) CDR1 with an amino acid sequence of GME, CDR2 with an amino acid sequence as set forth in SEQ ID NO: 19, and CDR3 with an amino acid sequence as set forth in SEQ ID NO: 20;
(8) CDR1 with an amino acid sequence of GME, CDR2 with an amino acid sequence as set forth in SEQ ID NO: 22, and CDR3 with an amino acid sequence as set forth in SEQ ID NO: 23;
(9) CDR1 with an amino acid sequence of GME, CDR2 with an amino acid sequence as set forth in SEQ ID NO: 25, and CDR3 with an amino acid sequence as set forth in SEQ ID NO: 26;
(10) CDR1 with an amino acid sequence of GME, CDR2 with an amino acid sequence as set forth in SEQ ID NO: 28, and CDR3 with an amino acid sequence as set forth in SEQ ID NO: 29;
(11) CDR1 with an amino acid sequence of GME, CDR2 with an amino acid sequence as set forth in SEQ ID NO: 31, and CDR3 with an amino acid sequence as set forth in SEQ ID NO: 32;
(12) CDR1 with an amino acid sequence of GQE, CDR2 with an amino acid sequence as set forth in SEQ ID NO: 34, and CDR3 with an amino acid sequence as set forth in SEQ ID NO: 35;
(13) CDR1 with an amino acid sequence of GQE, CDR2 with an amino acid sequence as set forth in SEQ ID NO: 37, and CDR3 with an amino acid sequence as set forth in SEQ ID NO: 38;
(14) CDR1 with an amino acid sequence of GQE, CDR2 with an amino acid sequence as set forth in SEQ ID NO: 40, and CDR3 with an amino acid sequence as set forth in SEQ ID NO: 41; or
(15) CDR1 with an amino acid sequence of GQE, CDR2 with an amino acid sequence as set forth in SEQ ID NO: 43, and CDR3 with an amino acid sequence as set forth in SEQ ID NO: 44.

10. The high-affinity anti-human PD-L1 single domain antibody according to claim 9, wherein the amino acid sequence of the complementary determining region (CDR) of the high-affinity anti-human PD-L1 single domain antibody is group (12) or group (15):
(12) CDR1 with an amino acid sequence of GQE, CDR2 with an amino acid sequence as set forth in SEQ ID NO: 34, and CDR3 with an amino acid sequence as set forth in SEQ ID NO: 35; or
(15) CDR1 with an amino acid sequence of GQE, CDR2 with an amino acid sequence as set forth in SEQ ID NO: 43, and CDR3 with an amino acid sequence as set forth in SEQ ID NO: 44.

11. The high-affinity anti-human PD-L1 single domain antibody according to claim 9, wherein an amino acid sequence of the high-affinity anti-human PD-L1 single domain antibody is as set forth in SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42 or SEQ ID NO: 45.

12. The high-affinity anti-human PD-L1 single domain antibody according to claim 11, wherein the amino acid sequence of the high-affinity anti-human PD-L1 single domain antibody is as set forth in SEQ ID NO: 36 or SEQ ID NO: 45.

13. A nucleic acid encoding the high-affinity anti-human PD-L1 single domain antibody according to claim 9.

14. A recombinant vector comprising the nucleic acid according to claim 13.

15. A transformant comprising the nucleic acid according to claim 13 or the recombinant vector according to claim 14.

16. A pharmaceutical composition comprising the high-affinity anti-human PD-L1 single domain antibody according to claim 9, and/or the nucleic acid according to claim 13, and/or the recombinant vector according to claim 14, and/or the transformant according to claim 15.

17. A use of the high-affinity anti-human PD-L1 single domain antibody according to claim 9, and/or the nucleic acid according to claim 13, and/or the recombinant vector according to claim 14, and/or the transformant according to claim 15, and/or the pharmaceutical composition according to claim 16 in a preparation of a drug for treating or alleviating tumors.


SEQUENCE LISTING (70 sequences)

SEQ ID NO: 1 - protein, 16 aa, synthetic construct
GITAGGSAYYTDTVKG

SEQ ID NO: 2 - protein, 7 aa, synthetic constuct
QYGPNWY

SEQ ID NO: 3 - protein, 113 aa, synthetic constuct
QVQLVESGGGLVQPGGSLRLSCAASARSINGQEWYRQAPGERRELVAGITAGGSAYYTDTVKGRFTISRDNAENTGYLQMNSLSPDDTAVYYCRRQYGPNWYWGQGTQVTVSS

SEQ ID NO: 4 - protein, 16 aa, synthetic constuct
GITSGGSAYYTDTVKG

SEQ ID NO: 5 - protein, 7 aa, synthetic constuct
QYGPNWY

SEQ ID NO: 6 - protein, 113 aa, synthetic constuct
QVQLVESGGGLVQPGGSLRLSCAASARSINGMEWYRQAPGERRELVAGITSGGSAYYTDTVKGRFTISRDNAENTGYLQMNSLSPDDTAVYYCRRQYGPNWYWGQGTQVTVSS

SEQ ID NO: 7 - protein, 16 aa, synthetic constuct
GITAGGSAYYTDTPKG

SEQ ID NO: 8 - protein, 7 aa, synthetic constuct
QYGPNWY

SEQ ID NO: 9 - protein, 113 aa, synthetic constuct
QVQLVESGGGLVQPGGSLRLSCAASARSINGMEWYRQAPGERRELVAGITAGGSAYYTDTPKGRFTISRDNAENTGYLQMNSLSPDDTAVYYCRRQYGPNWYWGQGTQVTVSS

SEQ ID NO: 10 - protein, 16 aa, synthetic constuct
GITAGGSAYYTDTYKG

SEQ ID NO: 11 - protein, 7 aa, synthetic constuct
QYGPNWY

SEQ ID NO: 12 - protein, 113 aa, synthetic constuct
QVQLVESGGGLVQPGGSLRLSCAASARSINGMEWYRQAPGERRELVAGITAGGSAYYTDTYKGRFTISRDNAENTGYLQMNSLSPDDTAVYYCRRQYGPNWYWGQGTQVTVSS

SEQ ID NO: 13 - protein, 16 aa, synthetic constuct
GITAGGSAYYTDTSKG

SEQ ID NO: 14 - protein, 7 aa, synthetic constuct
QYGPNWY

SEQ ID NO: 15 - protein, 113 aa, synthetic constuct
QVQLVESGGGLVQPGGSLRLSCAASARSINGMEWYRQAPGERRELVAGITAGGSAYYTDTSKGRFTISRDNAENTGYLQMNSLSPDDTAVYYCRRQYGPNWYWGQGTQVTVSS

SEQ ID NO: 16 - protein, 16 aa, synthetic constuct
GITAGGSAYYTDTTKG

SEQ ID NO: 17 - protein, 7 aa, synthetic constuct
QYGPNWY

SEQ ID NO: 18 - protein, 113 aa, synthetic constuct
QVQLVESGGGLVQPGGSLRLSCAASARSINGMEWYRQAPGERRELVAGITAGGSAYYTDTTKGRFTISRDNAENTGYLQMNSLSPDDTAVYYCRRQYGPNWYWGQGTQVTVSS

SEQ ID NO: 19 - protein, 16 aa, synthetic constuct
GITAGGSAYYTDTHKG

SEQ ID NO: 20 - protein, 7 aa, synthetic constuct
QYGPNWY

SEQ ID NO: 21 - protein, 113 aa, synthetic constuct
QVQLVESGGGLVQPGGSLRLSCAASARSINGMEWYRQAPGERRELVAGITAGGSAYYTDTHKGRFTISRDNAENTGYLQMNSLSPDDTAVYYCRRQYGPNWYWGQGTQVTVSS

SEQ ID NO: 22 - protein, 16 aa, synthetic constuct
GITAGGSAYYTDTKKG

SEQ ID NO: 23 - protein, 7 aa, synthetic constuct
QYGPNWY

SEQ ID NO: 24 - protein, 113 aa, synthetic constuct
QVQLVESGGGLVQPGGSLRLSCAASARSINGMEWYRQAPGERRELVAGITAGGSAYYTDTKKGRFTISRDNAENTGYLQMNSLSPDDTAVYYCRRQYGPNWYWGQGTQVTVSS

SEQ ID NO: 25 - protein, 16 aa, synthetic constuct
GITAGGSAYYTDTLKG

SEQ ID NO: 26 - protein, 7 aa, synthetic constuct
QYGPNWY

SEQ ID NO: 27 - protein, 113 aa, synthetic constuct
QVQLVESGGGLVQPGGSLRLSCAASARSINGMEWYRQAPGERRELVAGITAGGSAYYTDTLKGRFTISRDNAENTGYLQMNSLSPDDTAVYYCRRQYGPNWYWGQGTQVTVSS

SEQ ID NO: 28 - protein, 16 aa, synthetic constuct
GITAGGSAYYTDTVKG

SEQ ID NO: 29 - protein, 7 aa, synthetic constuct
QFGPNWY

SEQ ID NO: 30 - protein, 113 aa, synthetic constuct
QVQLVESGGGLVQPGGSLRLSCAASARSINGMEWYRQAPGERRELVAGITAGGSAYYTDTVKGRFTISRDNAENTGYLQMNSLSPDDTAVYYCRRQFGPNWYWGQGTQVTVSS

SEQ ID NO: 31 - protein, 16 aa, synthetic constuct
GITAGGSAYYTDTVKG

SEQ ID NO: 32 - protein, 7 aa, synthetic constuct
QVGPNWY

SEQ ID NO: 33 - protein, 113 aa, synthetic constuct
QVRLVESGGGLVQPGGSLRLSCAASARSINGMEWYRQAPGERRELVAGITAGGSAYYTDTVKGRFTISRDNAENTGYLQMNSLSPDDTAVYYCRRQVGPNWYWGQGTQVTVSS

SEQ ID NO: 34 - protein, 16 aa, synthetic constuct
GITAGGSAYYTDTVKG

SEQ ID NO: 35 - protein, 7 aa, synthetic constuct
QFGPNWY

SEQ ID NO: 36 - protein, 113 aa, synthetic constuct
QVQLVESGGGLVQPGGSLRLSCAASARSINGQEWYRQAPGERRELVAGITAGGSAYYTDTVKGRFTISRDNAENTGYLQMNSLSPDDTAVYYCRRQFGPNWYWGQGTQVTVSS

SEQ ID NO: 37 - protein, 16 aa, synthetic constuct
GITAGGSAYYTDTVKG

SEQ ID NO: 38 - protein, 7 aa, synthetic constuct
QVGPNWY

SEQ ID NO: 39 - protein, 113 aa, synthetic constuct
QVQLVESGGGLVQPGGSLRLSCAASARSINGQEWYRQAPGERRELVAGITAGGSAYYTDTVKGRFTISRDNAENTGYLQMNSLSPDDTAVYYCRRQVGPNWYWGQGTQVTVSS

SEQ ID NO: 40 - protein, 16 aa, synthetic constuct
GITAGGSAYYTDTHKG

SEQ ID NO: 41 - protein, 7 aa, synthetic constuct
QFGPNWY

SEQ ID NO: 42 - protein, 113 aa, synthetic constuct
QVQLVESGGGLVQPGGSLRLSCAASARSINGQEWYRQAPGERRELVAGITAGGSAYYTDTHKGRFTISRDNAENTGYLQMNSLSPDDTAVYYCRRQFGPNWYWGQGTQVTVSS

SEQ ID NO: 43 - protein, 16 aa, synthetic constuct
GITAGGSAYYTDTHKG

SEQ ID NO: 44 - protein, 7 aa, synthetic constuct
QVGPNWY

SEQ ID NO: 45 - protein, 113 aa, synthetic constuct
QVQLVESGGGLVQPGGSLRLSCAASARSINGQEWYRQAPGERRELVAGITAGGSAYYTDTHKGRFTISRDNAENTGYLQMNSLSPDDTAVYYCRRQVGPNWYWGQGTQVTVSS

SEQ ID NO: 46 - DNA, 24 bp, synthetic constuct
  source 1..24: mol_type = other DNA
caactccgccccattgacgcaaat

SEQ ID NO: 47 - DNA, 25 bp, synthetic constuct
  source 1..25: mol_type = other DNA
aaatgtaaacccgctggcggcacag

SEQ ID NO: 48 - DNA, 48 bp, synthetic constuct
  source 1..48: mol_type = other DNA
ctgtgccgccagcgggtttacatttgcctttaggcactatgtgatggg

SEQ ID NO: 49 - DNA, 48 bp, synthetic constuct
  source 1..48: mol_type = other DNA
ctgtgccgccagcgggtttacatttgactttaggcactatgtgatggg

SEQ ID NO: 50 - DNA, 48 bp, synthetic constuct
  source 1..48: mol_type = other DNA
ctgtgccgccagcgggtttacatttgagtttaggcactatgtgatggg

SEQ ID NO: 51 - DNA, 48 bp, synthetic constuct
  source 1..48: mol_type = other DNA
ctgtgccgccagcgggtttacatttttctttaggcactatgtgatggg

SEQ ID NO: 52 - DNA, 48 bp, synthetic constuct
  source 1..48: mol_type = other DNA
ctgtgccgccagcgggtttacatttggctttaggcactatgtgatggg

SEQ ID NO: 53 - DNA, 48 bp, synthetic construct
  source 1..48: mol_type = other DNA
ctgtgccgccagcgggtttacatttcactttaggcactatgtgatggg

SEQ ID NO: 54 - DNA, 48 bp, synthetic constuct
  source 1..48: mol_type = other DNA
ctgtgccgccagcgggtttacatttatctttaggcactatgtgatggg

SEQ ID NO: 55 - DNA, 48 bp, synthetic constuct
  source 1..48: mol_type = other DNA
ctgtgccgccagcgggtttacatttaagtttaggcactatgtgatggg

SEQ ID NO: 56 - DNA, 48 bp, synthetic constuct
  source 1..48: mol_type = other DNA
ctgtgccgccagcgggtttacatttctgtttaggcactatgtgatggg

SEQ ID NO: 57 - DNA, 48 bp, synthetic constuct
  source 1..48: mol_type = other DNA
ctgtgccgccagcgggtttacatttatgtttaggcactatgtgatggg

SEQ ID NO: 58 - DNA, 48 bp, synthetic constuct
  source 1..48: mol_type = other DNA
ctgtgccgccagcgggtttacatttaactttaggcactatgtgatggg

SEQ ID NO: 59 - DNA, 48 bp, synthetic constuct
  source 1..48: mol_type = other DNA
ctgtgccgccagcgggtttacatttccctttaggcactatgtgatggg

SEQ ID NO: 60 - DNA, 48 bp, synthetic constuct
  source 1..48: mol_type = other DNA
ctgtgccgccagcgggtttacatttcagtttaggcactatgtgatggg

SEQ ID NO: 61 - DNA, 48 bp, synthetic constuct
  source 1..48: mol_type = other DNA
ctgtgccgccagcgggtttacatttcgctttaggcactatgtgatggg

SEQ ID NO: 62 - DNA, 48 bp, synthetic constuct
  source 1..48: mol_type = other DNA
ctgtgccgccagcgggtttacattttcctttaggcactatgtgatggg

SEQ ID NO: 63 - DNA, 48 bp, synthetic constuct
  source 1..48: mol_type = other DNA
ctgtgccgccagcgggtttacatttgtgtttaggcactatgtgatggg

SEQ ID NO: 64 - DNA, 48 bp, synthetic constuct
  source 1..48: mol_type = other DNA
ctgtgccgccagcgggtttacattttggtttaggcactatgtgatggg

SEQ ID NO: 65 - DNA, 48 bp, synthetic constuct
  source 1..48: mol_type = other DNA
ctgtgccgccagcgggtttacattttactttaggcactatgtgatggg

SEQ ID NO: 66 - DNA, 24 bp, synthetic constuct
  source 1..24: mol_type = other DNA
acacggtacgtgctgttgtactgc

SEQ ID NO: 67 - DNA, 24 bp, synthetic constuct
  source 1..24: mol_type = other DNA
gtcctcctgactggggtgagggcc

SEQ ID NO: 68 - DNA, 24 bp, synthetic constuct
  source 1..24: mol_type = other DNA
agttttgtcagcagatttgggctc

SEQ ID NO: 69 - DNA, 339 bp, synthetic constuct
  source 1..339: mol_type = other DNA
caggtgcagctggtggaaagtggcggcggcctggtgcagcccggaggaagcctgagactgagctgcgccgccagcgccagaagcatcaacggcatggagtggtatagacaggccccaggagagagaagagagctggtggccggaatcaccgctggcggcagcgcttactacaccgacaccgtgaagggaagatttaccatcagcagagacaacgccgagaacacaggatacctgcagatgaacagcctgtctcccgatgacaccgccgtgtactactgcagaaggcaatatggtcccaattggtattggggccaggggacccaggtgaccgtgagcagc

SEQ ID NO: 70 - protein, 113 aa, synthetic constuct
QVQLVESGGGLVQPGGSLRLSCAASARSINGMEWYRQAPGERRELVAGITAGGSAYYTDTVKGRFTISRDNAENTGYLQMNSLSPDDTAVYYCRRQYGPNWYWGQGTQVTVSS